(19) United States
(12) Patent Application Publication
(10) Pub. No.: US 2008/0145872 A1
(43) Pub. Date: Jun. 19, 2008

(54) Expression of HLA proteins on non human cells

(76) Inventor: William H. Hildebrand, Edmond, OK (US)

Correspondence Address: DUNLAP CODDING & ROGERS, P.C., PO BOX 16370, OKLAHOMA CITY, OK, 73113, US

(21) Appl. No.: 12/009,262

(22) Filed: Jan. 17, 2008

Related U.S. Application Data
(63) Continuation-in-part of application No. 11/099,283, filed on Apr. 4, 2005.
Application No. 11/099,283 is a continuation of application No. 10/022,066, filed on Dec. 18, 2001, now abandoned.
Application No. 10/022,066 is a continuation-in-part of application No. 09/465,321, filed on Dec. 17, 1999, now abandoned.
Application No. 09/465,321 is a continuation-in-part of application No. 09/974,366, filed on Oct. 10, 2001.
(60) Provisional application No. 60/880,838, filed on Jan. 17, 2007.
(60) Provisional application No. 60/256,410, filed on Dec. 18, 2000.
(60) Provisional application No. 60/256,409, filed on Dec. 18, 2000.
(60) Provisional application No. 60/327,907, filed on Oct. 9, 2001.

(51) Int. Cl. G01N 33/53; C12N 5/06; C12P 21/04; C07K 1/00; G01N 33/567

(52) U.S. Cl. 435/7.21; 435/325; 435/69.1; 436/547; 530/402

(57) ABSTRACT

The present invention is directed to a composition that includes a functionally active, individual HLA trimolecular complex expressed on the surface of an immortalized, non-human cell line. Methods of obtaining high expression of the transferred HLA molecule, as well as assays that utilize such recombinant cell line as a platform, are also disclosed. Methods of purifying HLA from such recombinant cell line and methods of using the purified HLA to detect or remove anti-HLA antibodies are also disclosed.

### CROSS REFERENCE TO RELATED APPLICATIONS

This application claims benefit under 35 U.S.C. 119(e) of U.S. Ser. No. 60/880,838, filed Jan. 17, 2007. This application is also a continuation-in-part of U.S. Ser. No. 11/099,283, filed Apr. 4, 2005; which is a continuation of U.S. Ser. No. 10/022,066, filed Dec. 18, 2001, now abandoned; which claims priority under 35 U.S.C. § 119(e) of provisional U.S. Ser. No. 60/256,410, filed Dec. 18, 2000; provisional U.S. Ser. No. 60/256,409, filed Dec. 18, 2000; and provisional U.S. Ser. No. 60/327,907, filed Oct. 9, 2001. Said application U.S. Ser. No. 10/022,066 is also a continuation-in-part of U.S. Ser. No. 09/465,321, filed Dec. 17, 1999, now abandoned; and is also a continuation-in-part of U.S. Ser. No. 09/974,366, filed Oct. 10, 2001.

The entire contents of each of the above-referenced patents and patent applications are hereby expressly incorporated herein by reference.

### STATEMENT REGARDING FEDERALLY SPONSORED RESEARCH OR DEVELOPMENT

Not Applicable.

### BACKGROUND OF THE INVENTION

1. Field of the Invention

The present invention relates generally to a methodology of expression of human HLA proteins on non-human cells as well as to methods of utilizing such non-human cells having HLA proteins expressed thereon.

2. Description of the Background Art

Human cells express on their surface an incredibly large number of membrane-bound proteins, all of which display individual properties and physiological functions. From this large array of surface cell proteins, a number of clinical procedures require characterization of the human major histocompatibility complex (MHC) class I and II membrane-bound molecules. The human MHC class I and class II molecules are known as human leukocyte antigens, or HLA. The HLA class I and class II molecules are responsible for presenting peptide antigens to receptors located on the surface of T-lymphocytes, Natural Killer Cells (NK), and possibly other immune effector and regulatory cells. Display of peptide antigens on the MHC I and MHC II molecules are the basis for the recognition of “self vs. non-self” and the onset of important immune responses such as transplant rejection, graft-versus-host-disease, autoimmune disease, and healthy anti-viral and anti-bacterial immune responses.

Class I and class II HLA molecules differ from person to person. Each person expresses a different complement of class I and class II on the surface of their cells. For transplant purposes it is important to determine which of the multiple HLA expressed on a cell are recognized by the antibodies of another individual. Anti-HLA antibodies can lead to hyperacute organ rejection. It is often difficult to determine which of many HLA are recognized by antibodies because sera can have antibodies to non-HLA proteins, multiple HLA molecules, and sera may crossreact among different HLA molecules. With many human proteins, many HLA proteins, antibodies to multiple human proteins, and antibodies crossreactive to various HLA proteins, it can be difficult when screening patients for organ transplantation to ascertain which of the many HLA in the population, and expressed on an organ to be transplanted, are recognized by antibodies. Antibodies to HLA proteins may also lead to problems during the transfusion of blood products, whereby antibodies in the blood of the blood donor may react with the HLA class I and class II antigens of the recipient of the blood product. Antibodies in the blood product that recognize the recipient's HLA may lead to transfusion related acute lung injury (TRALI).

Class I MHC molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptide antigens bound and presented by class II HLA are derived from extracellular foreign antigens, such as products of bacteria that multiply outside of cells, wherein such products include protein toxins secreted by the bacteria or any other bacterial protein to which the human immune system might respond in a protective manner. In this manner, class II molecules convey information regarding the existence of pathogens in extracellular spaces that are accessible to the cell displaying the class II molecule. Class II HLA expressing cells then present peptide antigens derived from the extracellular antigen/bacteria to immune effector cells, including but not limited to, CD4⁺ helper T cells, thereby helping to eliminate such pathogens. The elimination of such pathogens is accomplished by both helping B cells make antibodies against microbes, as well as toxins produced by such microbes, and by activating macrophages to destroy ingested microbes.

Class I and class II HLA molecules exhibit extensive polymorphism generated by systematic recombinatorial and point mutation events; as such, hundreds of different HLA types exist throughout the world's population, resulting in substantial immunologic diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, and because of their tremendous diversity, large quantities of individual HLA proteins are required in order to effectively study transplantation, autoimmunity disorders, and for vaccine development.

However, there has been no readily available source of individual HLA molecules. The quantities of HLA protein available have been small and typically consist of a mixture of different HLA molecules. Production of HLA molecules traditionally involves growth and lysis of cells expressing multiple HLA molecules. Ninety percent of the population is heterozygous at each of the HLA loci; codominant expression results in multiple HLA proteins expressed at each HLA locus. To purify native class I or class II molecules from human cells requires time-consuming and cumbersome purification methods in order to separate individual class I or class II HLA molecules away from other HLA proteins expressed by the cell, and since each cell typically expresses multiple surface-bound HLA class I or class II molecules, HLA purification results in a mixture of many different HLA class I or class II molecules. When performing experiments using such a mixture of HLA molecules or performing experiments using a cell having multiple surface-bound HLA molecules, interpretation of results cannot directly distinguish between the different HLA molecules, and one cannot be certain that any particular HLA molecule is responsible for a given result. Therefore, prior to the present invention, a need existed in the art for a method of producing substantial quantities of individual HLA class I or class II molecules so that they can be readily purified and isolated independent of other HLA class I or class II molecules. Such individual HLA molecules, when provided in sufficient quantity and purity as described herein, provides a powerful tool for studying and measuring immune responses.

The fact that HLA class I contains three genes of interest: HLA-A, B, and C, while HLA class II contains multiple gene products as well including, DRA, DRB, DPA, DPB, DQA, and DQB, must be taken into consideration. To add to the complexity of the system, these proteins are polymorphic and are expressed in a heterozygous fashion, meaning that each cell expresses one molecule form from the mother and another from the father, leading to the expression of many different MHC molecules on each cell surface. Furthermore, antibodies have a difficult time discriminating among various class II—this complicates the serologic selection of cells that express large numbers of a particular HLA class II, and this complicates the purification of a given class II HLA protein.

Identification of class I and class II HLA molecules on the cell surface by serological methods is difficult and complex. The cross reactivity between these molecules and the high background given by other surface-bound molecules that have similarities to the MHC system make this task unreliable.

Therefore, there exists a need in the art for improved methods of expressing individual HLA class I and class II molecules. There exists a need for selecting for high expression of an individual HLA protein, and there exists a need for purifying a specific HLA protein without copurification of mixtures of HLA proteins. The present invention solves this need by expressing individual human HLA class I and HLA class II molecules on non-human cells. Such system will have many advantages over the existing procedures of HLA serologic characterization.

### BRIEF DESCRIPTION OF THE DRAWINGS

FIG. 1 illustrates expression of MHC II on NS-1. Shaded area, untransfected NS-1; green line, human T2 cells, known not to express MHC II; Blue line and red line, THP-1 and U937 respectively, human macrophage-like cell lines known to express MHC II; black line, NS-1 double transfected with DRA1*0101 and DRB1*0101.

FIG. 2 illustrates NS-1 transfectants expressing human MHC II. Shaded area, untransfected NS-1; black line, DRA1*0101/DRB1*0401; blue line, DRA1*0101/DRB1*0801; red line, DRA1*0101/DRB1*1101; and green line, DRA1*0101/DRB1*0102

FIG. 3 illustrates plasma from 4 individuals pre-tested as DR1, tested again against NS-1 transfected with human MHC II. Shaded area, unstained cells; black line, cells stained with secondary antibody only; dashed line, fully stained NS-1 transfected with only DRA1; red line, NS-1 DRA1*0101/DRB1*0101; blue line, NS-1 DRA1*0101/DRB1*0401; green line, DRA1*0101/DRB1*0801; and purple line, DRA1*0101/DRB1*1101.

FIG. 4 illustrates the selection of high expression of NS-1 transfected with DRA1*0101. NS-1 cells were analyzed by flow cytometry for their expression of DRA*0101. Column 1, untransfected NS-1 cells; column 2, initial transfer and expression of DRA*0101; columns 3-5: NS-1 cells transfected with DRA1*0101 after one, two or three sorting steps, respectively.

FIG. 5 illustrates the selection of high expression of NS-1 transfected with DRB1*0101. NS-1 cells were analyzed by flow cytometry for their expression of DRB1*0401/DRA1*0101. Column 1, untransfected NS-1 cells; column 2, column 2 from FIG. 4 (used to establish a threshold); column 3, class II alpha/beta/peptide expression on human cell line 721.221; column 4, initial transfection of DRB1*0401 into the DRA1*0101 positive cells shown in column 2; columns 5 and 6: NS-1 cells transfected with DRA1*0101/DRB1*0401 after one or two sorting steps, respectively.

FIG. 6 graphically represents a sandwich ELISA platform for the detection of specific Anti-HLA class II antibodies in human sera, in accordance with the present invention.

FIG. 7 show typical reactivity patterns of specific human sera with the Class II molecule DRB1*0401 in the ELISA platform of FIG. 6.

FIG. 8 illustrates a sera screen similar to FIG. 7, except using a different clone of HLA-DRB*0401.

FIG. 9 graphically represents a direct detection approach for the detection of specific Anti-HLA class II antibodies in human sera, in accordance with the present invention.

FIG. 10 illustrates ELISA results of HLA-DRB1*0401 directly coated in the ELISA platform of FIG. 9.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The terms “isolated polynucleotide” and “isolated nucleic acid segment” as used herein shall mean a polynucleotide of genomic, cDNA, or synthetic origin or some combination thereof, which by virtue of its origin the “isolated polynucleotide” or “isolated nucleic acid segment” (1) is not associated with all or a portion of a polynucleotide in which the “isolated polynucleotide” or “isolated nucleic acid segment” is found in nature, (2) is operably linked to a polynucleotide which it is not linked to in nature, or (3) does not occur in nature as part of a larger sequence.

The term “isolated protein” referred to herein means a protein of genomic, cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or, (4) does not occur in nature.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “MHC” as used herein will be understood to refer to the Major Histocompability Complex, which is defined as a set of gene loci specifying major histocompatibility antigens. The term “HLA” as used herein will be understood to refer to Human Leukocyte Antigens, which is defined as the major histocompatibility antigens found in humans. As used herein, “HLA” is the human form of “MHC”.

The terms “MHC class I light chain” and “MHC class I heavy chain” as used herein will be understood to refer to portions of the MHC class I molecule. Structurally, class I molecules are heterodimers comprised of two noncovalently bound polypeptide chains, a larger “heavy” chain (α) and a smaller “light” chain (β-2-microglobulin or β2m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3^{rd} extracellular domain of the molecule contains the recognition site for the CD8 protein on the CTL; this interaction serves to stabilize the contact between the T cell and the APC. The invariant light chain (12 kDa), encoded outside the MHC on chromosome 15, consists of a single, extracellular polypeptide. The terms “MHC class I light chain”, “β-2-microglobulin”, and “β2m” may be used interchangeably herein. Association of the class I heavy and light chains is required for expression of class I molecules on cell membranes.

Like MHC class I molecules, class II molecules are also heterodimers, but in this case consist of two nearly homologous α and β chains, both of which are encoded in the MHC. The class II MHC molecules are membrane-bound glycoproteins, and both the α and β chains contain external domains, a transmembrane anchor segment, and a cytoplasmic segment. Each chain in a class II molecule contains two external domains: the 33-kDa α chain contains α₁ and α₂ external domains, while the 28-kDa β chain contains β₁ and β₂ external domains. The membrane-proximal α₂ and β₂ domains, like the membrane-proximal 3^{rd} extracellular domain of class I heavy chain molecules, bear sequence homology to the immunoglobulin-fold domain structure. The membrane-distal domain of a class II molecule is composed of the α₁ and β₁ domains, which form an antigen-binding cleft for processed peptide antigen. The peptides presented by class II molecules are derived from extracellular proteins (not cytosolic intracellular peptide antigens as in class I); hence, the MHC class II-dependent pathway of antigen presentation is called the endocytic or exogenous pathway. Loading of class II molecules must still occur inside the cell; extracellular proteins are endocytosed, digested in lysosomes, and bound by the class II MHC molecule prior to the molecule's migration to the plasma membrane. Because the peptide-binding groove of MHC class II molecules is open at both ends while the corresponding groove on class I molecules is closed at each end, the peptides presented by MHC class II molecules are longer, generally between 13 and 24 amino acid residues long. Like class I HLA, the peptides that bind to class II molecules often have internal conserved “motifs’, but unlike class I-binding peptides, they lack conserved motifs at the carboxyl-terminal end, since the open ended binding cleft allows a bound peptide to extend from both ends.

The term “trimolecular complex” as used herein will be understood to refer to the MHC heterodimer associated with a peptide. An “MHC class I trimolecular complex” or “HLA class I trimolecular complex” will be understood to include the class I heavy and light chains associated together and having a peptide displayed in an antigen binding groove thereof. The terms “MHC class II trimolecular complex” and “HLA class II trimolecular complex” will be understood to include the class II alpha and beta chains associated together and having a peptide displayed in an antigen binding groove thereof.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)2 and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

The term “biological sample” as used herein will be understood to include, but not be limited to, serum, tissue, blood, cerebrospinal fluid, tears, saliva, lymph, dialysis fluid, organ or tissue culture derived fluids, and fluids extracted from physiological tissues. The term “biological sample” as used herein will also be understood to include derivatives and fractions of such fluids, as well as combinations thereof. For example, the term “biological sample” will also be understood to include complex mixtures.

Turning now to the present invention, the methods disclosed herein are directed to the expression of individual human HLA class I and HLA class II molecules on non-human cells and methods of purifying and/or using same. Such systems will have many advantages over the existing procedures of HLA serologic characterization, including, but not limited to, those discussed in the following paragraphs.

One advantage of the present invention over the prior art is the ability to select for non-human cells that express a high number of one specific surface-bound human HLA class I or class II molecule. The one specific HLA molecule, or an alpha or beta chain thereof, will be transferred into the non-human cells. Because the non-human cells do not express other competing HLA, the present invention allows for the selection of high expression of a single human HLA protein without background interference from other human proteins that may be serologically crossreactive. This will facilitate the establishment of cell lines that express high amounts of a single class I or class II HLA protein.

Class I and class II highly expressed HLA proteins can then be purified without interference from other human proteins that might interfere with the HLA purification due to crossreactivity or similarity to the human HLA protein to be characterized. The present invention allows for the utilization of a human-specific HLA purification scheme that will not be impacted by other human proteins because they are not found in the non-human cells that have been transfected to express HLA.

Since the present invention provides control of the environment where the HLA molecule is expressed (i.e., the HLA molecule will be the only human molecule on a non-human mammalian background), many other experiments can be performed in order to better understand the biochemical and biophysical properties of the human MHC. The present invention essentially eliminates background noise that would otherwise interfere with studying how manipulations change expression and purification of an individual HLA protein. Changes can be made in the HLA protein, the gene promoter, etc., and the impact of these changes on the HLA protein in question can be assessed without interference from other non-modified HLA.

Expression of human HLA class I and class II on non-human cells also allows the clear detection of antibodies that have formed against human HLA, even when antibodies may exist in the serum at low concentration or that may have a low avidity for the HLA molecules. Non-human cells expressing human HLA molecules can be used in cell arrays (Stephan et al., 2002), where human serum could be tested against a panel of non-human cells that are each expressing different HLA molecules. Thus, non-human cells that are not reactive to serum can be identified, and then a single HLA protein can be expressed in these non-human cells, thus facilitating the clear and precise serologic characterization of this HLA protein.

Because the non-human cells will only express a single human molecule, the background given by the binding antibody to a given molecule will be low, and could be completely eliminated by subtracting the background of the HLA non-transfected non-human cells, from the binding observed when using the non-human cell expressing the given HLA molecule.

Other systems that use human cells to produce HLA typically purify the HLA and then put the HLA on a surface meant to resemble the cell from which HLA was extracted. This allows the HLA to be the only human protein available, but these systems struggle to purify the desired HLA away from other HLA. In the present invention, HLA will be the only human protein available on the cell. Therefore, the HLA may never need to be extracted from the non-human cell, purified, concentrated, and adhered to a cell-like surface. All of these extraction/purification/concentration/adherence steps can impact HLA protein yield and can alter the HLA protein conformation such that it loses some or all of its native structure and serologic recognition. Expressing an HLA protein on a non-human cell obviates extraction, purification, adherence, and the like.

The non-human cells expressing the HLA protein may be used as an HLA platform themselves rather than requiring extraction of the HLA protein. Therefore, one embodiment of the present invention is a composition comprising a recombinant, non-human cell having at least one functionally active, individual HLA trimolecular complex on a surface thereof.

The term “HLA protein” as used herein will be understood to refer to any HLA molecule, complex thereof or fragment thereof that is capable of being expressed on a surface of a non-human cell. Examples of HLA proteins that may be utilized in accordance with the present invention include, but are not limited to, an HLA class I trimolecular complex, an HLA class II trimolecular complex, an HLA class II α chain and an HLA class II chain. Specific examples of HLA class II α and/or β proteins that may be utilized in accordance with the present invention include, but are not limited to, those encoded at the following gene loci: HLA-DRA; HLA-DRB1; HLA-DRB3,4,5; HLA-DQA; HLA-DQB; HLA-DPA; and HLA-DPB.

The term “non-human cell” as used herein will be understood to refer to any cell capable of expressing a recombinant HLA protein (as defined herein above) on a surface thereof. Therefore, any “non-human cell” utilized in accordance with the present invention must contain the necessary machinery and transport proteins required for expression of MHC/HLA proteins and/or MHC/HLA trimolecular complexes on a surface of such cell. “Non-human cells” utilized in accordance with the present invention must have (A) machinery for chaperoning and loading MHC/HLA proteins, such as class I and class II proteins; and (B) such machinery must be able to interact and work with human HLA proteins, such as class I and class II proteins. Not all cells express class II MHC protein; only professional immune cells such as but not limited to dendritic cells (DC), macrophages, B cells, and the like express class II proteins. Therefore, when it is desired to express class II HLA protein in a non-human cell, such non-human cell must express class II MHC for that species and contain the appropriate machinery for interacting and working with both that species' class II MHC as well as human class II HLA. However, the present invention also includes the use of cells of other lineages that have been induced to express class II MHC, such as but not limited to, cytokines, cells that have been subjected to mutagenesis, and the like.

In addition, “non-human cells” utilized in accordance with the present invention must not only express class II MHC for that species and be capable of expressing human class II HLA, but the endogenous class II MHC present in such cell must also not cross react with antibodies against the human class II HLA. This allows for high human HLA class II expression when the antibodies utilized do not cross react with class II from the species of the non-human cell. In addition, this prevents cross reaction of sera from the non-human class II MHC when using the HLA class II expressing non-human cell as a platform for presenting transfected human class II HLA.

The term “non-human cell” as used herein refers to immortalized non-human cell lines and does not include animals or primary cells.

Examples of “non-human cells” that may be utilized in accordance with the present invention include, but are not limited to, mouse DC lines, macrophage lines, and B cell lines. Specific examples include, but are not limited to, murine B cell lines such as NS-1, A20 and EL4.

The non-human cells expressing HLA can be repeatedly selected for high expression of the HLA protein put into the cell without interference from other human proteins.

The non-human cells can be selected for high expression of the selected HLA protein, and then the HLA protein can be extracted in large quantities due to high expression.

The purification of HLA extracted from non-human cells will be much less complex and require fewer purification steps because other competing HLA/human proteins will not need to be subtracted or otherwise selected out during the purification process. Purification of the selected HLA protein will be simplified.

The present invention therefore provides a method of purifying HLA, wherein the method comprises providing a recombinant vector comprising at least one nucleic acid segment encoding alpha and beta chains of at least one HLA molecule (as defined herein above). An immortalized, non-human cell line is transformed with the recombinant vector to produce a non-human cell line having at least one functionally active, individual HLA trimolecular complex expressed on a surface thereof, wherein the functionally active, individual HLA trimolecular complex comprises alpha and beta chains having an endogenously loaded peptide displayed in an antigen binding groove formed by the alpha and beta chains. The functionally active, individual HLA trimolecular complex is then purified substantially away from other proteins such that the individual HLA trimolecular complex maintains the physical, functional and antigenic integrity of a native HLA trimolecular complex.

There are many purification methods available for the separation of macromolecules. To effectively resolve a crude mixture of substances, it may be necessary to use a combination of techniques. In most cases, a purification procedure will involve some chromatographic techniques.

Affinity chromatography occupies a unique place in separation technology since it is the only technique which enables purification of almost any biomolecule on the basis of its biological function or individual chemical structure. Affinity chromatography makes use of specific binding interactions that occur between molecules. It is a type of adsorption chromatography in which the molecule to be purified is specifically and reversibly adsorbed by a complementary binding substance (ligand) immobilized on an insoluble support (matrix). A single pass through an affinity column can achieve a 1,000-10,000 fold purification of ligand from a crude mixture. It is possible to isolate a compound in a form pure enough to obtain a single band upon SDS-polyacrylamide gel electrophoresis. Any component that has an interacting counterpart can be attached to a support and used for affinity purification.

Successful separation by affinity chromatography requires that a biospecific ligand is available and that it can be covalently attached to a chromatographic bed material called a matrix. It is important that the biospecific ligand (antibody, enzyme, receptor protein or other affinity reagent) retains its specific binding affinity for the substance of interest (antigen, substrate, or hormone). Methods must also include removing the bound material in active form with low pH, high pH, or high salt. The selection of the ligand for affinity chromatography is influenced by two factors. Firstly, the ligand should exhibit specific and reversible binding affinity for the substance to be purified. Secondly, it should have chemically modifiable groups, which allow it to be attached to the matrix without destroying its binding activity. The ligand should ideally have an affinity for the binding substance in the range 10-4 to 10-8 M in free solution.

In affinity chromatography, nonspecific proteins flow through the column while the specific protein is retained by the column. The protein is then eluted, and individual fractions are tested for specific-binding activity and purity. Several different approaches can be taken to allow efficient binding of antigens to immunoaffinity columns. Because the ligand/affinity reagent is not in solution, the time required for the ligand-matrix/antigen interaction will have different kinetics than soluble interactions. It will take considerably longer for equilibrium to be reached than for solution assays. Therefore, the binding protocol should maximize the degree of interaction. The recommended method is binding by passing the antigen solution down an antibody-matrix column, keeping the antigen in contact with the antibody for as long as possible. In this case, high-affinity antibodies will be significantly more efficient at removing the antigen from solution than low-affinity antibodies. Several small-scale columns can be used to determine the best conditions for binding and collecting the antigen.

Therefore, the purification step of the methods of the present invention may comprise affinity chromatography utilizing, for example but not by way of limitation, an antibody specific for the HLA trimolecular complex, or an anchoring moiety attached to the HLA trimolecular complex. Examples of anchoring moieties and chromatography affinity reagents that may be used in accordance with the present invention include, but are not limited to, (i) a hisitidine tag, utilized with affinity reagents such as nickel, copper and/or combinations thereof; (ii) a biotinylation signal peptide, utilized with affinity reagents such as avidin or streptavidin; and (iii) a VLDLr tail or FLAG tail, utilized with affinity reagents that recognize the VLDLr or FLAG tails. However, it is to be understood that the present invention is not limited to the specific combinations of anchoring moieties and chromatography affinity reagents described above. Rather, any combination of anchoring moiety and chromatography affinity reagent known in the art may be used in accordance with the present invention and therefore also fall within the scope thereof.

The cell surface is the natural environment for HLA, and using the transfected non-human cell as an HLA platform will provide the most natural environment for assays that require or benefit from HLA in its native conformation. The native conformation of HLA expressed on non-human cells is especially useful for screening antibodies in serologic assays because anti-HLA antibodies are especially sensitive to changes in the native conformation of the HLA protein. The expression of HLA on non-human cells provides a “conformationally natural platform” for antibody screening assays.

Therefore, the present invention is also directed to a method for detecting the presence of anti-HLA antibodies in a biological sample. In such method, an HLA platform is provided; the HLA platform comprises a recombinant, non-human cell having at least one functionally active, individual HLA trimolecular complex on a surface thereof, wherein the non-human cell is from an immortalized, non-human cell line and contains the necessary machinery and transport proteins required for expression of MHC proteins on a surface of the non-human cell. The individual HLA trimolecular complex comprises alpha and beta chains having an endogenously loaded peptide in an antigen binding groove formed by the alpha and beta chains. A biological sample is also provided and reacted with the HLA platform, whereby antibodies specific for the HLA protein will bind to the HLA platform. The HLA platform is then washed to remove unbound portions of the biological sample, followed by reacting the HLA platform with means for detecting anti-HLA antibodies. It is then determined that anti-HLA antibodies specific for the functionally active, individual HLA trimolecular complex are present in the biological sample if the means for detecting anti-HLA antibodies is positive. The means for detecting an anti-HLA antibody may be a labeled antibody that recognizes at least one of anti-human IgG, IgM and IgA antibodies.

Non-human cells expressing HLA can also be utilized for antibody removal assays. Humans with antibodies to a particular HLA could have their serum dialyzed, such as but not limited to, through a hollow fiber bioreactor dialysis chamber containing non-human cells expressing the desired HLA. Antibodies to the HLA would be absorbed or removed from the patient and would therefore no longer be a barrier to transplantation

Therefore, the present invention is also directed to methods of removing anti-HLA antibodies from a biological sample. In the method, an HLA platform as described above is provided. A biological sample is also provided and reacted with the HLA platform, whereby antibodies specific for the HLA trimolecular complex will bind to the HLA platform and are thus removed from the biological sample.

HLA expressing non-human cells could be “fixed” such that they are no longer alive. In this instance fixed cells would express HLA in a native conformation at the surface of the fixed cells. Fixed cells could be immobilized on any number of platforms (see for example but not by way of limitation, Stephan et al., 2002), and sera or antibodies could be tested for their recognition of the fixed and immobilized cells. Alternatively, HLA expressing non-human cells could be fixed and tested for antibody reactivity in a liquid phase assay such as flow cytometry.

As HLA proteins present peptide ligands that provoke anti-viral, anti-bacterial, autoimmune, anti-transplant (minor histocompatibility antigens), regulatory, and anti-cancer immune responses, individual HLA could be characterized for their ability to present cancer related, virus related, bacterial, and self (autoimmune and regulatory) peptides in non-human cells. HLA proteins expressed in mice are known to present peptide antigens as they do in humans (Rohrlich et al., 2003), and HLA molecules expressed in mice could be used to discover therapeutic and diagnostic peptide epitopes for cancer and for bacterial antigens taken into the non-human cell expressing the human HLA.

In other embodiments of the present invention, HLA trimolecular complexes purified away from the non-human cells may be utilized. The HLA trimolecular complexes may be purified as described herein above or by any other methods known to a person having ordinary skill in the art.

The present invention also includes a method in which a substrate is provided, wherein the substrate is selected from the group consisting of a well, a bead, a membrane, a microtiter plate, a matrix, a pore, plastic, glass, a polymer, a polysaccharide, nylon, nitrocellulose, a paramagnetic compound, and combinations thereof. Next, a functionally active, individual HLA trimolecular complex purified substantially away from other proteins such that the individual HLA trimolecular complex maintains the physical, functional and antigenic integrity of a native HLA trimolecular complex is provided. The functionally active, individual HLA trimolecular complex may be purified as described above or by any other method known in the art. The functionally active, individual HLA trimolecular complex comprises alpha and beta chains with an endogenously loaded peptide displayed in an antigen binding groove formed by the alpha and beta chains. the functionally active, individual HLA trimolecular complex is then directly or indirectly linked to the substrate, wherein the conformation of the functionally active, individual HLA trimolecular complex is maintained when the functionally active, individual HLA trimolecular complex is linked to the substrate.

The functionally active, individual HLA trimolecular complex may be directly attached to the substrate, or the HLA trimolecular complex may be indirectly attached to the substrate via an anchoring moiety selected from the group consisting of an antibody to the functionally active, individual HLA trimolecular complex and a tail or tag attached to the functionally active, individual HLA trimolecular complex. The tail or tag may be a histidine tag, a biotinylation signal peptide, a VLDLr tail or a FLAG tail.

In addition, the method of detecting and/or removing anti-HLA antibodies discussed herein above may also be performed utilizing purified HLA. The present invention also includes a method for detecting the presence of anti-HLA antibodies in a biological sample. Such method includes providing a substrate and a functionally active, individual HLA trimolecular complex purified substantially away from other proteins such that the individual HLA trimolecular complex maintains the physical, functional and antigenic integrity of a native HLA trimolecular complex. The functionally active, individual HLA trimolecular complex comprises alpha and beta chains with an endogenously loaded peptide displayed in an antigen binding groove formed by the alpha and beta chains. The functionally active, individual HLA trimolecular complex is then directly or indirectly linked to the substrate, wherein the conformation of the functionally active, individual HLA trimolecular complex is maintained when the functionally active, individual HLA trimolecular complex is linked to the substrate. A biological sample is then reacted with the substrate having the functionally active, individual HLA trimolecular complex linked thereto, and the substrate is washed to remove unbound portions of the biological sample. The substrate having the functionally active, individual HLA trimolecular complex linked thereto is then reacted with means for detecting anti-HLA antibodies, and it is determined that anti-HLA antibodies specific for the individual HLA trimolecular complex are present in the biological sample if the means for detecting anti-HLA antibodies is positive.

The functionally active, individual HLA trimolecular complex may be directly attached to the substrate, or the HLA trimolecular complex may be indirectly attached to the substrate via an anchoring moiety selected from the group consisting of an antibody to the functionally active, individual HLA trimolecular complex and a tail or tag attached to the functionally active, individual HLA trimolecular complex. The tail or tag may be a histidine tag, a biotinylation signal peptide, a VLDLr tail or a FLAG tail.

The substrate may be a solid support selected from the group consisting of a well, a bead, a membrane, an ELISA plate, and a matrix.

The present invention also includes a method for removing anti-HLA antibodies from a biological sample. The method includes providing a substrate and a functionally active, individual HLA trimolecular complex purified substantially away from other proteins such that the individual HLA trimolecular complex maintains the physical, functional and antigenic integrity of a native HLA trimolecular complex. The functionally active, individual HLA trimolecular complex comprises alpha and beta chains with an endogenously loaded peptide displayed in an antigen binding groove formed by the alpha and beta chains. The functionally active, individual HLA trimolecular complex is then directly or indirectly linked to the substrate, wherein the conformation of the functionally active, individual HLA trimolecular complex is maintained when the functionally active, individual HLA trimolecular complex is linked to the substrate. A biological sample is then reacted with the substrate having the functionally active, individual HLA trimolecular complex linked thereto, whereby antibodies specific for the HLA trimolecular complex are removed from the biological sample.

The functionally active, individual HLA trimolecular complex may be directly attached to the substrate, or the HLA trimolecular complex may be indirectly attached to the substrate via an anchoring moiety selected from the group consisting of an antibody to the functionally active, individual HLA trimolecular complex and a tail or tag attached to the functionally active, individual HLA trimolecular complex. The tail or tag may be a histidine tag, a biotinylation signal peptide, a VLDLr tail or a FLAG tail.

The substrate may be a solid support selected from the group consisting of a well, a bead, a membrane, an ELISA plate, and a matrix.

In conclusion, the expression of human HLA molecules on non-human cells can be a powerful tool in facilitating the identification and characterization of serologic antibodies directed towards HLA molecules. Serologic background reactivity to other human proteins, especially to other HLA, can be greatly reduced and eliminated. Such anti-HLA antibodies are naturally occurring in all individuals and must be identified and characterized prior to allogeneic grafting, transplantation and the transfer of blood products. In addition, the lack of human HLA on non-human cells allows for the selection of non-human cells expressing high levels of human HLA. High levels of human HLA facilitate specificity and sensitivity in downstream diagnostic and therapeutic applications.

Examples are provided hereinbelow. However, the present invention is to be understood to not be limited in its application to the specific experimentation, results and laboratory procedures. Rather, the Examples are simply provided as one of various embodiments and are meant to be exemplary, not exhaustive.

### EXAMPLES

### Example 1

### Transfection of Class II HLA-DR Molecules on Mouse NS-1 Cells

The methods began by transfecting either the HLA-DR alpha chain (DRA*0101) or the HLA-DR beta chain (DRB*0101) into NS-1 cells. It was then shown by flow cytometry that the transfected alpha or beta chain could be expressed on the cell surface. Once it was known that an alpha or beta chain could be expressed alone, these alpha and beta chains were selected by flow cytometry for high expression. Next, the corresponding chain was then transfected so that DRB*0101 and DRA*0101 were expressed together. Flow cytometry was then utilized to show that the class II alpha and beta chains were being co-expressed on the surface of NS-1. It was found that co-expression of a human alpha and beta chain increased expression logarithmically; the human alpha and beta chain complement each other to dramatically increase expression on mouse NS-1 cells. Therefore, it was shown that HLA DR alpha and beta chains can be expressed on mouse cells and that HLA alpha/beta coexpression greatly enhances HLA class II expression as measured by antibodies specific for human class 11 molecules.

Given that the mice cells transfected with human class II were expressing HLA DR alpha and beta chains, next it was tested whether these HLA-DR expressing mouse cells were recognized by human sera specific for particular human class II molecules. Sera that is specific for HLA-DRA1*0101/DRB1*0101 was obtained from a clinical laboratory. Using flow cytometry, this DRB1*0101 sera was tested against mouse cells expressing DRA1*0101/DRB1*0101, DRA1*0101/DRB1*0401, DRA1*0101/DRB1*0801, and DRA1*0101/DRB1*1101. It was found that the sera for DRB1*0101 stained the DRB1*0101 cells significantly more than mouse cells expressing DRB1*0401, DBR1*0801, and DRB1*1101. These data show that the methods of the present invention express HLA class II proteins on mouse cells, and that this HLA class II is specifically recognized by sera for HLA class II. The protein is therefore antigenically correct, and the mouse cells expressing the human class II protein may be utilized as a platform for class II applications.

### Example 2

### Expression of Human MHC II on Mouse Cell Line

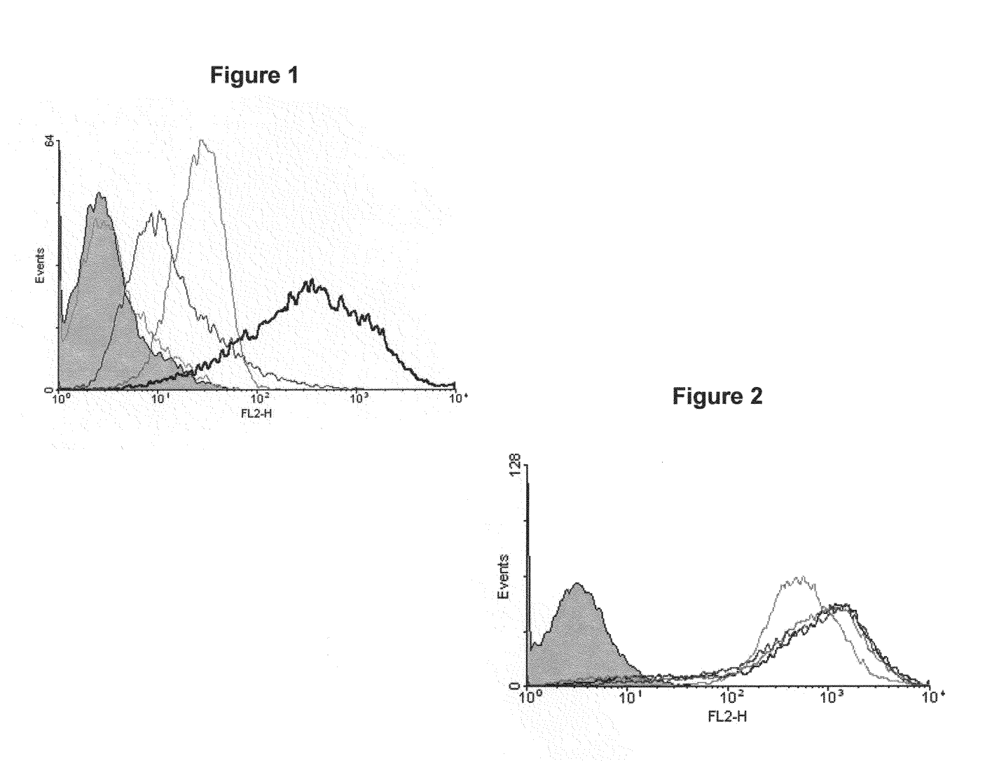

Murine cell line NS-1 was first transfected with DRA1*0101 expressed in the pcDNA3.1/G418 vector (Invitrogen). After proper antibiotic selection, transfectants were identified by flow cytometry using mAb mouse anti-HLA-DR L243 (BioLegends) F(ab)₂ fragment PE-goat anti-mouse IgG. High expressers for DRA1 were single cell cloned and then double transfected with the human MHC II beta chain expressed in the pcDNA3.1/Zeocin vector (Invitrogen). After the second antibiotic selection, the transfectants were stained with mAb mouse anti-HLA-DRB TDR31.1 (ABCAM) and then with F(ab)₂ fragment PE-goat anti-mouse IgG, and compared to untransfected NS-1 and to different human cell lines (FIG. 1).

Fifteen transfectants have been obtained, all of which are high expressers of human MHC II (FIG. 2).

The presence of alpha and beta chains as well as the corroboration of the allele placed in each transfectant was performed by PCR-allele typing.

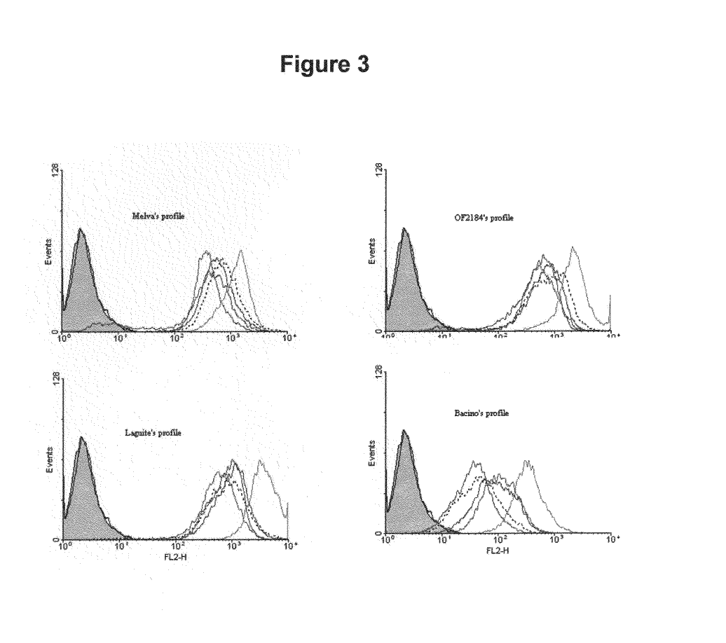

Transfectants expressing human MHC II were also tested against 4 sera pre-typed DR1 before by the Ochsner Transplant Center, Histocompatibility & Immunogenetics Laboratory. The results are shown in FIG. 3, demonstrating that the transfectants made are specific in being recognized by specific human antibodies present in the plasma.

### Example 3

### Selection of High Expression HLA Class II HLA-DR on Mouse NS-1 Cells

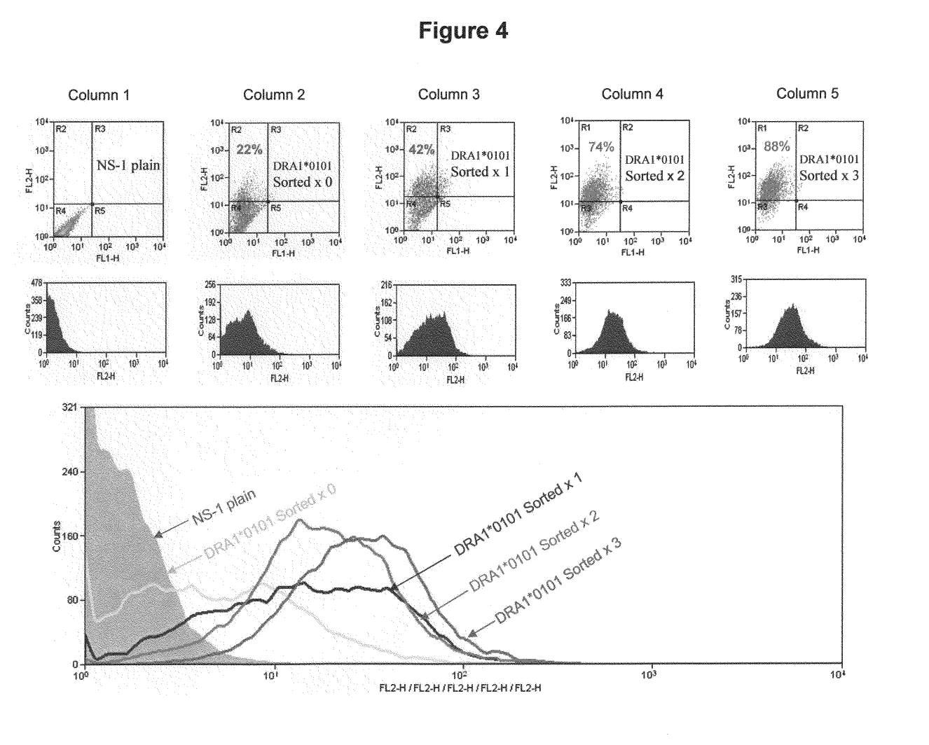

FIG. 4 illustrates analysis of NS-1 transfected with DRA1*0101. NS-1 cells were analyzed by flow cytometry for their expression of DRA1*0101. In column 1, the untransfected NS-1 cells show no DRA1*0101 class II expression. In column 2, the initial transfer and expression of DRA1*0101 showed that 22% of the cells were positive. In column three, after one sort, 42% were positive for DRA1*0101 class II expression; 74% were positive after two sorts in column 4, and 88% were positive after 3 sorts in column five. These data show that flow cytometry can be utilized to select for the high expression of the transferred class II molecule.

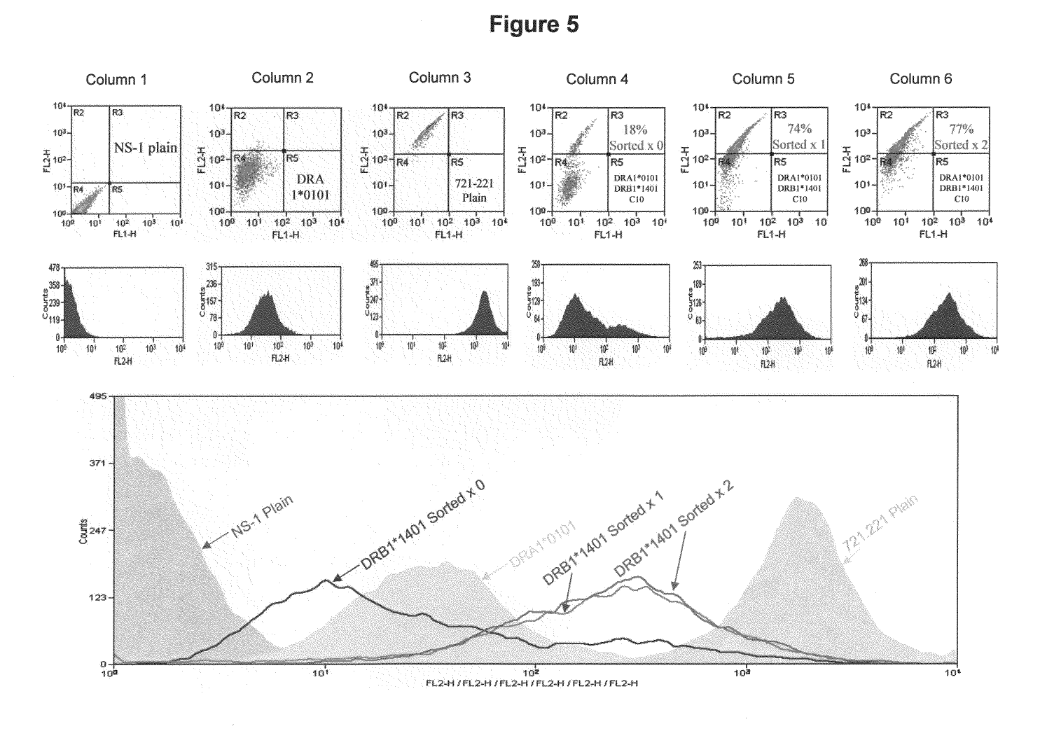

FIG. 5 illustrates analysis of NS-1 transfected with DRB1*0101. NS-1 cells were analyzed by flow cytometry for their expression of DRB1*0401/DRA1*0101. In column 1, the untransfected NS-1 cells show no class II expression. In column 2, the expression of the previously transfected DRA1*0101 is used to establish a threshold. As a point of comparison, class II alpha/beta/peptide expression on the human cell line 721.221, known to express plentiful class 11, is demonstrated in column three. In column four, the initial transfection of DRB1*0101 into the DRA1*0101 positive cells results in 18% of the NS-1 cells rising above the DRA1*0101-only threshold. After one or two sorts (columns 5 and 6, respectively), the alpha/beta class II expression rises to 74% and 77%, respectively. These data show that high expression of selected HLA class II alpha/beta combinations can be achieved using the technique described herein.

### Example 4

### Affinity Purification of HLA Class II from Recombinant, Non-Human Cell Lines

The Class II molecules produced in accordance with the present invention were subjected to affinity purification. The affinity purification protocol is provided herein below.

Upon completing a roller-bottle run generating approximately 10¹⁰ cells, cells were washed in PBS followed by the extraction of HLA class II complexes in MHC lysis buffer consisting of 1% NP-40 in PBS and a protease inhibitor cocktail (Sigma). To increase the efficiency of extraction, the samples were sonicated by applying ten 5-second bursts over a time period of 30 minutes. After centrifugation of the cell debris, the crude extract was affinity purified using the DR class II antibody L243 coupled to a Sepharose 4B matrix (Amersham; Piscataway, N.J.). Extracts were applied to the column using a peristaltic pump system (Amersham) with a speed of 1 ml/min at 4° C. After the column was extensively washed with phosphate-buffered saline (PBS), bound HLA molecules were eluted with 0.1 M glycine (pH 11.0) and immediately neutralized by addition of 1 M Tris-HCI, pH 7.0 to preserve the activity of the eluted molecules. Under these conditions, HLA molecules were recovered with a 60-70% yield. Purified molecules were buffer exchanged with PBS at pH 7.2 and concentrated using 10 kDa cut-off MACROSEP® centrifugal concentrators (Pall Filtron; Northborough, Mass.). The final product was filter-sterilized, and stored at 4° C. until further use.

### Example 5

### Sandwich ELISA Platform for Sera Screening Analyses for Detection of Specific Anti-HLA Class II Antibodies

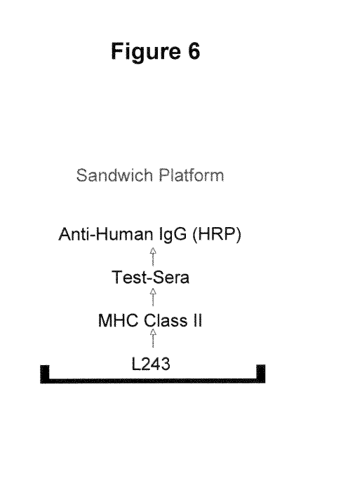

FIG. 6 graphically represents the experimental procedure for the detection of specific Anti-HLA class II antibodies in human sera, utilizing HLA class II trimolecular complexes produced in accordance with the present invention. The presented sandwich ELISA platform uses an anti-Class II antibody (L243), capturing a class II allele of interest and presenting it to the test sera. Anti-HLA class II antibodies with the sera will specifically bind to those MHC molecules and can then be detected by an anti-human IgG antibody.

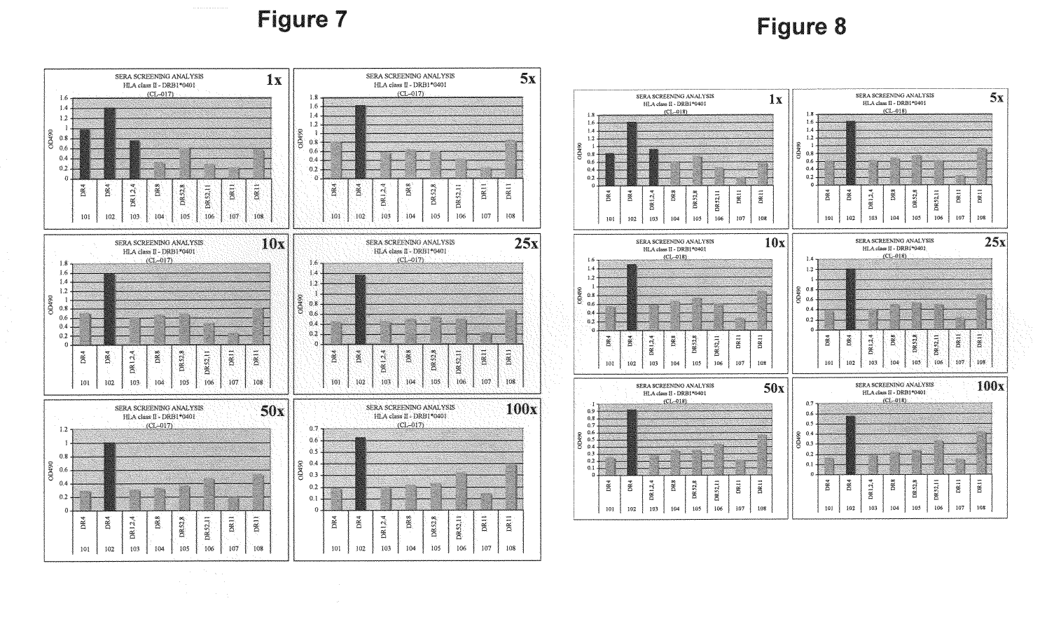

FIG. 7 demonstrates the typical reactivity patterns of specific human sera with the Class II molecule DRB1*0401 (CL-017). Eight human sera containing anti-class II antibodies with different amounts and specificity were tested. Results clearly demonstrate as expected, that the DRB1*0401 molecule only recognizes DR4 sera and none of the other antibody-species presented by the sera. In addition, it is also evident that sera 102 has a very high titer and can be detected up to 100× diluted, whereas the presence of anti-DR4 in sera 101 and 103 are rather low.

In FIG. 8, the sera experiments were repeated with another batch of HLA-DRB1*0401 (CL-018), thereby confirming the repeatability and consistency of the HLA molecule as well the technique applied.

### Example 6

### Direct Approach ELISA Platform for Sera Screening Analyses for Detection of Specific Anti-HLA Class II Antibodies

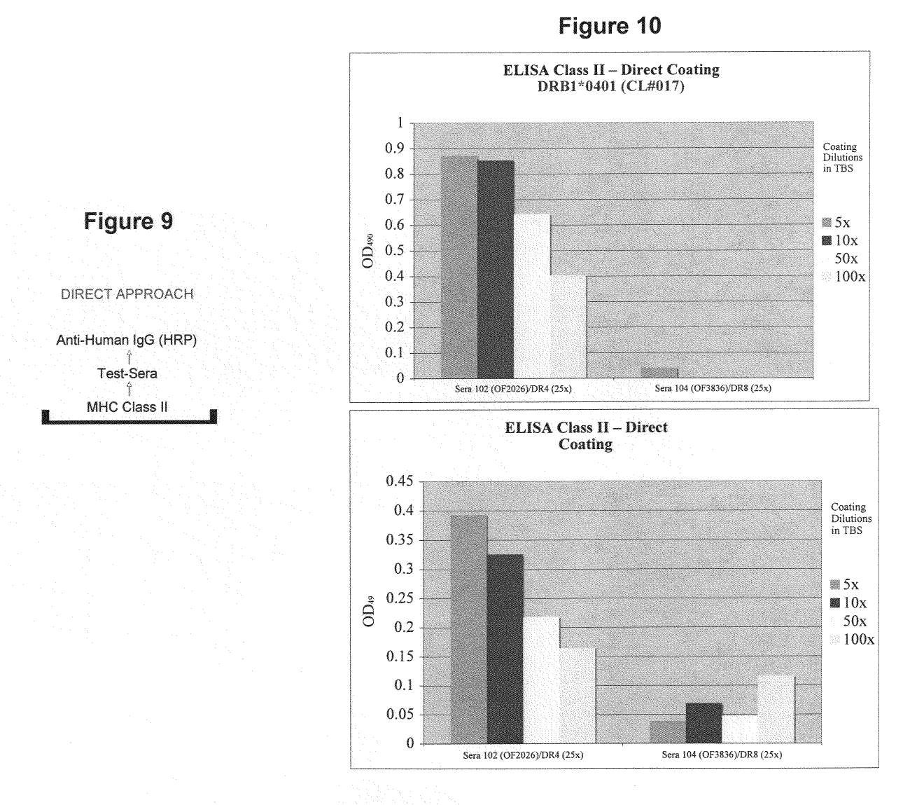

As an alternative to the sandwich method, a direct detection approach can be applied for the detection of specific Anti-HLA class II antibodies in human sera. This technique is graphically illustrated in FIG. 9. Experimentally, no capturing antibody is applied rather MHC class II molecules are directly coated to a plate. After this initial step, the procedure is identical using an anti-human IgG antibody to detect anti-HLA class II antibodies in human sera.

In FIG. 10, the reactivity of directly coated HLA DRB1*0401 molecules of two different batches were tested using a positive (DR4) and a negative (DR8) sera. In both cases, only the positive sera reacted in a titer-dependant manner, whereas the negative sera remained negative. A background of only 0.1 OD was notated.

Thus, in accordance with the present invention, there has been provided a methodology for producing HLA molecules on non-human cells that fully satisfies the objectives and advantages set forth herein above. Although the invention has been described in conjunction with the specific drawings, experimentation, results and language set forth herein above, it is evident that many alternatives, modifications, and variations will be apparent to those skilled in the art. Accordingly, it is intended to embrace all such alternatives, modifications and variations that fall within the spirit and broad scope of the invention.

### REFERENCES

The following references, to the extent that they provide exemplary procedural or other details supplementary to those set forth herein, are specifically incorporated herein by reference.

Stephan, J P et al. Development of a frozen cell array as a high-throughput approach for cell-based analysis. 2002. American Journal of Pathology 161: 787-797.

Pierre-Simon Rohrlich et al. HLA-B*0702 transgenic H-2 KbDb double-knockout mice: phenotypical and functional characterization in response to influenza virus. 2003. International Immunology, 15:765-772.

What is claimed is:

1. A composition, comprising:
a recombinant, non-human cell having at least one functionally active, individual HLA trimolecular complex on a surface thereof, wherein the non-human cell is from an immortalized, non-human cell line and contains the necessary machinery and transport proteins required for expression of MHC proteins on a surface of the non-human cell, and wherein the at least one functionally active, individual HLA trimolecular complex comprises alpha and beta chains having an endogenously loaded peptide displayed in an antigen binding groove formed by the alpha and beta chains.

2. The composition of claim 1, wherein the HLA trimolecular complex is selected from the group consisting of HLA-DRA, HLA-DRB1, HLA-DRB3, HLA-DRB4, HLA-DRB5, HLA-DQA, HLA-DQB, HLA-DPA, and HLA-DPB.

3. The composition of claim 1, wherein the non-human cell expresses endogenous class II MHC trimolecular complexes on a surface thereof.

4. The composition of claim 3, wherein the endogenous class II MHC present in the non-human cell does not cross react with antibodies against class II HLA.

5. A method of purifying HLA, wherein the method comprises the steps of:
providing a recombinant vector comprising at least one nucleic acid segment encoding alpha and beta chains of at least one HLA molecule;
transforming an immortalized, non-human cell line with the recombinant vector to produce a non-human cell line having at least one functionally active, individual HLA trimolecular complex expressed on a surface thereof, wherein the at least one HLA trimolecular complex comprises alpha and beta chains having an endogenously loaded peptide displayed in an antigen binding groove formed by the alpha and beta chains; and
purifying the functionally active, individual HLA trimolecular complex substantially away from other proteins such that the individual HLA trimolecular complex maintains the physical, functional and antigenic integrity of a native HLA trimolecular complex.

6. The method of claim 5, wherein the HLA molecule is selected from the group consisting of HLA-DRA, HLA-DRB1, HLA-DRB3, HLA-DRB4, HLA-DRB5, HLA-DQA, HLA-DQB, HLA-DPA, and HLA-DPB.

7. The method of claim 5 wherein, in the step of transforming an immortalized, non-human cell line, the immortalized, non-human cell line expresses endogenous class II MHC trimolecular complexes on a surface thereof.

8. The method of claim 7, wherein the endogenous class II MHC present in the immortalized, non-human cell line does not cross react with antibodies against class II HLA.

9. The method of claim 5, wherein the step of purifying the HLA trimolecular complex comprises affinity chromatography.

10. The method of claim 9, wherein the affinity chromatography utilizes an antibody specific for the HLA trimolecular complex.

11. The method of claim 9, wherein the HLA trimolecular complex comprises an anchoring moiety, and wherein at least one of:
(a) the anchoring moiety is a histidine tag, and wherein the affinity chromatography utilizes an affinity reagent selected from the group consisting of nickel, copper and combinations thereof;
(b) the anchoring moiety is a biotinylation signal peptide, and wherein the affinity chromatography utilizes an affinity reagent selected from avidin or streptavidin; and
(c) the anchoring moiety is a VLDLr or FLAG tail, and wherein the affinity chromatography utilizes an affinity reagent that recognizes the VLDLr or FLAG tail.

12. A method for detecting the presence of anti-HLA antibodies in a biological sample, comprising the steps of:
providing an HLA platform comprising a recombinant, non-human cell having at least one functionally active, individual HLA trimolecular complex on a surface thereof, wherein the non-human cell is from an immortalized, non-human cell line and contains the necessary machinery and transport proteins required for expression of MHC proteins on a surface of the non-human cell, and wherein the at least one functionally active, individual HLA trimolecular complex comprises alpha and beta chains having an endogenously loaded peptide displayed in an antigen binding groove formed by the alpha and beta chains;
providing a biological sample;
reacting the biological sample with the HLA platform, whereby antibodies specific for the HLA protein will bind to the HLA platform;
washing the HLA platform to remove unbound portions of the biological sample;
reacting the HLA platform with means for detecting anti-HLA antibodies; and
determining that anti-HLA antibodies specific for the functionally active, individual HLA trimolecular complex are present in the biological sample if the means for detecting anti-HLA antibodies is positive.

13. The method of claim 12, wherein the HLA trimolecular complex is selected from the group consisting of HLA-DRA, HLA-DRB1, HLA-DRB3, HLA-DRB4, HLA-DRB5, HLA-DQA, HLA-DQB, HLA-DPA and HLA-DPB.

14. The method of claim 12 wherein, in the step of providing an HLA platform, the immortalized, non-human cell line expresses endogenous MHC class II trimolecular complexes on a surface thereof.

15. The method of claim 12 wherein, in the step of providing a biological sample, the biological sample is selected from the group consisting of serum, tissue, blood, cerebrospinal fluid, tears, saliva, lymph, dialysis fluid, organ or tissue culture derived fluids, fluids extracted from physiological tissues, and combinations thereof.

16. A method for removing anti-HLA antibodies from a biological sample, comprising the steps of:
providing an HLA platform comprising a recombinant, non-human cell having at least one functionally active, individual HLA trimolecular complex on a surface thereof, wherein the non-human cell is from an immortalized, non-human cell line and contains the necessary machinery and transport proteins required for expression of MHC proteins on a surface of the non-human cell, and wherein the at least one functionally active, individual HLA trimolecular complex comprises alpha and beta chains having an endogenously loaded peptide displayed in an antigen binding groove formed by the alpha and beta chains;
providing a biological sample; and
reacting the biological sample with the HLA platform, whereby antibodies specific for the HLA trimolecular complex are removed from the biological sample.

17. The method of claim 16, wherein the HLA trimolecular complex is selected from the group consisting of HLA-DRA, HLA-DRB1, HLA-DRB3, HLA-DRB4, HLA-DRB5, HLA-DQA, HLA-DQB, HLA-DPA, and HLA-DPB.

18. The method of claim 16 wherein, in the step of providing an HLA platform, the immortalized, non-human cell line expresses endogenous MHC class II trimolecular complexes on a surface thereof.

19. The method of claim 16 wherein, in the step of providing a biological sample, the biological sample is selected from the group consisting of serum, tissue, blood, cerebrospinal fluid, tears, saliva, lymph, dialysis fluid, organ or tissue culture derived fluids, fluids extracted from physiological tissues, and combinations thereof.

20. A method, comprising the steps of:
providing a substrate selected from the group consisting of a well, a bead, a membrane, a microtiter plate, a matrix, a pore, plastic, glass, a polymer, a polysaccharide, nylon, nitrocellulose, a paramagnetic compound, and combinations thereof;
providing a functionally active, individual HLA trimolecular complex purified substantially away from other proteins such that the individual HLA trimolecular complex maintains the physical, functional and antigenic integrity of a native HLA trimolecular complex, wherein the functionally active, individual HLA trimolecular complex comprises alpha and beta chains with an endogenously loaded peptide displayed in an antigen binding groove formed by the alpha and beta chains; and
directly or indirectly linking the functionally active, individual HLA trimolecular complex to the substrate, wherein the conformation of the functionally active, individual HLA trimolecular complex is maintained when the functionally active, individual HLA trimolecular complex is linked to the substrate.

21. The method of claim 20, wherein the HLA trimolecular complex is selected from the group consisting of HLA-DRA, HLA-DRB1, HLA-DRB3, HLA-DRB4, HLA-DRB5, HLA-DQA, HLA-DQB, HLA-DA, and HLA-DPB.

22. The method of claim 20, wherein the functionally active, individual HLA trimolecular complex is indirectly attached to the substrate via an anchoring moiety selected from the group consisting of an antibody to the functionally active, individual HLA trimolecular complex and a tail or tag attached to the functionally active, individual HLA trimolecular complex.

23. The method of claim 22, wherein the tail or tag is a histidine tag, a biotinylation signal peptide, a VLDLr tail or a FLAG tail.

24. A method for detecting the presence of anti-HLA antibodies in a biological sample, comprising the steps of:
providing a substrate;
providing a functionally active, individual HLA trimolecular complex purified substantially away from other proteins such that the individual HLA trimolecular complex maintains the physical, functional and antigenic integrity of a native HLA trimolecular complex, wherein the functionally active, individual HLA trimolecular complex comprises alpha and beta chains with an endogenously loaded peptide displayed in an antigen binding groove formed by the alpha and beta chains; and
directly or indirectly linking the functionally active, individual HLA trimolecular complex to the substrate, wherein the conformation of the functionally active, individual HLA trimolecular complex is maintained when the functionally active, individual HLA trimolecular complex is linked to the substrate;
providing a biological sample;
reacting the biological sample with the substrate having the functionally active, individual HLA trimolecular complex linked thereto;
washing the substrate to remove unbound portions of the biological sample;
reacting the substrate having the functionally active, individual HLA trimolecular complex linked thereto with means for detecting anti-HLA antibodies; and
determining that anti-HLA antibodies specific for the individual HLA trimolecular complex are present in the biological sample if the means for detecting anti-HLA antibodies is positive.

25. The method of claim 24, wherein the HLA trimolecular complex is selected from the group consisting of HLA-DRA, HLA-DRB1, HLA-DRB3, HLA-DRB4, HLA-DRB5, HLA-DQA, HLA-DQB, HLA-DPA and HLA-DPB.

26. The method of claim 24 wherein, in the step of providing a biological sample, the biological sample is selected from the group consisting of serum, tissue, blood, cerebrospinal fluid, tears, saliva, lymph, dialysis fluid, organ or tissue culture derived fluids, fluids extracted from physiological tissues, and combinations thereof.

27. The method of claim 24, wherein the functionally active, individual HLA trimolecular complex is indirectly attached to the substrate via an anchoring moiety selected from the group consisting of an antibody to the functionally active, individual HLA trimolecular complex and a tail or tag attached to the functionally active, individual HLA trimolecular complex.

28. The method of claim 27, wherein the tail or tag is a histidine tag, a biotinylation signal peptide, a VLDLr tail or a FLAG tail.

29. The method of claim 24 wherein, in the step of providing a substrate, the substrate is a solid support selected from the group consisting of a well, a bead, a membrane, an ELISA plate, and a matrix.

30. A method for removing anti-HLA antibodies from a biological sample, comprising the steps of:
providing a substrate;
providing a functionally active, individual HLA trimolecular complex purified substantially away from other proteins such that the individual HLA trimolecular complex maintains the physical, functional and antigenic integrity of a native HLA trimolecular complex, wherein the functionally active, individual HLA trimolecular complex comprises alpha and beta chains with an endogenously loaded peptide displayed in an antigen binding groove formed by the alpha and beta chains; and
directly or indirectly linking the functionally active, individual HLA trimolecular complex to the substrate, wherein the conformation of the functionally active, individual HLA trimolecular complex is maintained when the functionally active, individual HLA trimolecular complex is linked to the substrate;
providing a biological sample; and
reacting the biological sample with the substrate having the functionally active, individual HLA trimolecular complex linked thereto, whereby antibodies specific for the HLA trimolecular complex are removed from the biological sample.

31. The method of claim 30, wherein the HLA trimolecular complex is selected from the group consisting of HLA-DRA, HLA-DRB1, HLA-DRB3, HLA-DRB4, HLA-DRB5, HLA-DQA, HLA-DQB, HLA-DPA, and HLA-DPB.

32. The method of claim 30 wherein, in the step of providing a biological sample, the biological sample is selected from the group consisting of serum, tissue, blood, cerebrospinal fluid, tears, saliva, lymph, dialysis fluid, organ or tissue culture derived fluids, fluids extracted from physiological tissues, and combinations thereof.

33. The method of claim 30, wherein the functionally active, individual HLA trimolecular complex is indirectly attached to the substrate via an anchoring moiety selected from the group consisting of an antibody to the functionally active, individual HLA trimolecular complex and a tail or tag attached to the functionally active, individual HLA trimolecular complex.

34. The method of claim 33, wherein the tail or tag is a histidine tag, a biotinylation signal peptide, a VLDLr tail or a FLAG tail.

35. The method of claim 30 wherein, in the step of providing a substrate, the substrate is a solid support selected from the group consisting of a well, a bead, a membrane, an ELISA plate, and a matrix.
